(19) United States
(12) Patent Application Publication
(10) Pub. No.: US 2003/0034035 A1
(43) Pub. Date: Feb. 20, 2003

(54) Determining endotracheal tube placement using acoustic reflectometry

(75) Inventor: David T. Raphael, Valley Village, CA (US)

Correspondence Address: MCDERMOTT, WILL & EMERY (LOS ANGELES OFFICE), 2049 CENTURY PARK EAST, 34TH FLOOR, LOS ANGELES, CA, 90067-3208, US

(73) Assignee: Alfred E.Mann Institute for Biomedical Engineering at the University of Southern California

(21) Appl. No.: 10/218,480

(22) Filed: Aug. 14, 2002

Related U.S. Application Data
(60) Provisional application No. 60/312,283, filed on Aug. 14, 2001.

(51) Int. Cl. A61M 16/00

(52) U.S. Cl. 128/207.14

(57) ABSTRACT

Determining the placement of an endotracheal tube in a patient. The invention evaluates discontinuities in the medium surrounding the endotracheal tube, such as the airway, as a function of distance past an end of the endotracheal tube. Using a loudspeaker to generate sound waves, the sound waves propagate through a coiled wavetube, a connecting adapter, and an endotracheal tube, into the area of interest. With a processing system, reflected sound waves which return from the cavity back to a microphone within the wavetube are analyzed and an area-distance curve of the area in interest is constructed.

### CROSS-REFERENCE TO RELATED APPLICATIONS

This application claims priority to and incorporates by reference U.S. Provisional Application No. 60/312,283, filed Aug. 14, 2001, entitled “Miniature, Portable Acoustic Reflectometer.”

### BACKGROUND OF THE INVENTION

1. Field of the Invention

The invention relates to intubations and, more particularly, determining whether an endotracheal tube (“ETT”) has been correctly placed and positioned.

2. Description of Related Art

In the pre-hospital setting, the incidence of unrecognized esophageal intubations varies between 1.8-2.0%. Current methods that assist physicians in correctly performing an endotracheal intubation consist of direct visualization of the passage of the ETT through the vocal cords, fiberoptic bronchoscopy, and detection of expired carbon dioxide. Direct visualization may not always be possible because of certain patient conditions, particularly in difficult airway patients. Fiberoptic bronchoscopy may not always be available, especially in emergency settings outside the operating room. Detection of expired carbon dioxide may not be possible in patients such as those with cardiopulmonary arrest where there is no characteristic color change in the calorimetric device and/or a wave form is absent in a capnograph or spectrometer trace.

For pulseless patients, an esophageal detector device which relies upon application of suction to a device attached to the ETT is available. However, this device is sometimes slow, requiring up to thirty seconds for a determination. It is often not usable in infants less than 1 year of age, having a failure rate of approximately 25%. It can also generate false results when there is an airway obstruction, such as a foreign body, asthma, or a mediastinal mass compressing the trachea.

In patients with an adequate circulation, whether in the operating room or in the field, the detection of expired end-tidal carbon dioxide can be accomplished through use of either a capnograph or a single-use end-tidal colorimetric device. However, even these devices may present spurious results. For example, in a patient in cardiopulmonary arrest, in whom there is no pulmonary circulation, there may be no characteristic color change in the calorimetric device, and a wave form may be absent in a capnograph. It is the critical failure to recognize an esophageal intubation that continues to pose a serious safety problem, either in difficult airway patients in whom the endotracheal tube placement is indeterminate and for whom capnography is not available, or in a cardiac arrest patient for whom capnography is rendered useless.

Additionally, in patients requiring endotracheal intubation, the patency of the ETT lumen can often be compromised by the accumulation of mucus secretions that are more pronounced in patients with respiratory disease. Large mucus plugs can markedly reduce ventilatory flow and impair systemic oxygenation, and a complete obstruction, if undetected and unrelieved, will result in death. Upon attempted weaning from mechanical ventilation, the presence of mucus plugs also may impede spontaneous breathing by increasing the work required for breathing.

In adult patients, the internal diameter of ETTs used can range from 6.0 to 8.5 mm (often 8.0 mm for males and 7.0 for females). In pediatric patients up to six-months of age, the internal diameter of ETTs used ranges from 2.5-3.5 mm. In such narrow pediatric ETTs, the relative effect of mucus plugging is much more pronounced. In an ETT with a 3 mm internal diameter, a reduction by 1 mm represents a 66% decrease in diameter, an 89% reduction in cross-sectional area, and a decrease of airflow of greater than 98% at constant pressures. A larger proportion of the airflow through the narrowed tube will be turbulent rather than laminar, requiring even greater inspiratory pressures to maintain flow. These differences require maintenance of an inventory of ETTs of different diameters.

For intubated pediatric patients, a clinician or nurse is alerted to the problem of an obstructed ETT by the triggering of machine alarms whose limits have been exceeded, such as the pulse oximeter alarm (decrease in the oxygen saturation) or a ventilator alarm (possibly due to inadequate delivery of gas volume delivery or a rise in the circuit pressure gauge). The clinical diagnosis of ETT obstruction is made by failure of chest wall motion in response to attempted manual ventilation with a reservoir bag, and by the obstructed passage of a suction catheter through the ETT. Should the suctioning attempt fail to remove the cause of the obstruction (kink in the ETT, mucus plug, herniated ETT cuff), the ETT is replaced with a new one. A significant problem with this current method of clinical management of the obstructed ETT is that the required response must be immediate, with its associated stress and sense of urgency. Currently in the intensive care unit setting, there is no non-invasive device which can be used to perform a ‘quick look’ into the ETT in order to assess the possibility of ETT obstruction, and to simultaneously detect the extent and location of airway narrowing.

### SUMMARY OF THE INVENTION

The invention aids in the determination of whether an endotracheal intubation (endotracheal tube (“ETT”) in the trachea) has been done correctly, or whether an incorrect ETT placement has occurred (ETT in the esophagus). The invention also includes enhancements in acoustic reflectometer technology that are useful in medical applications, as well as others.

In one embodiment, the distal end of an ETT is placed through the mouth or nose of a patient and advanced into a cavity (either the trachea or the esophagus of the patient). Acoustic pulses are sent through a wavetube that is in acoustic communication with the ETT and the cavity. Reflections of the acoustic pulses from within the ETT, and more distally from within the cavity, travel back through the wavetube. A microphone in the wavetube measures the pressure amplitudes of the reflected acoustic pulses. Using a mathematical algorithm, the reflected pulse data are transformed into data representative of the cross-sectional areas in the ETT and the cavity throughout a range of corresponding distances beyond the distal end of the ETT. An image of this data in the form of an area-distance profile is displayed. The image is examined to determine the placement (correct tracheal or incorrect esophageal) and position (tracheal versus bronchial) of the distal end of the ETT within the patient.

In one embodiment, the distal end of the ETT ends up in the patient's esophagus. In alternative embodiments, the distal end of the ETT ends up in the trachea or in the bronchus. Each ending position of the ETT provides different identifiable and characteristic area-versus-distance profiles on the display. These distinctive area-distance profiles allow the acoustic differentiation as to the specific position and placement of the ETT.

In one embodiment, the Gopillaud-Ware-Aki mathematical algorithm is applied to the pressure amplitudes of the reflected pulses in order to produce an area-versus-distance profile on the display.

In one embodiment, oxygen is delivered through the ETT after determining the placement and positioning of the ETT.

In one embodiment, very short acoustic pulses are used, lasting a few milliseconds and with frequencies up to 10 KHz. In one embodiment, the acoustic pulses last less than two milliseconds.

In one embodiment, the wavetube and the ETT are a single tube.

In one embodiment, the invention includes an acoustic reflectometer instrument that is used to determine correct or incorrect placement and positioning of the ETT following an endotracheal intubation. The instrument includes an ETT, a wavetube for acoustic communication with the ETT, a sound generator, a sound receiver for receiving reflections of the sound within the wavetube, a processor that communicates with the sound receiver and transforms the reflections into data, and a display that communicates with the processor to display, for distances beyond the distal end of the ETT, an image of the cross-sectional area at any given axial distance into the studied cavity of the patient.

In one embodiment, the wavetube is made of silicon.

In one embodiment, the sound generator is a loudspeaker.

In one embodiment, the sound receiver is only one microphone.

In one embodiment, the sound receiver is two microphones.

In one embodiment, a connecting adapter connects the wavetube to the ETT.

In one embodiment, the wavetube and the ETT constitute a single tube.

In one embodiment, a battery powers the instrument.

In one embodiment, the invention includes an apparatus for use with an acoustic reflectometer. The apparatus includes a coiled wavetube, a sound generator, and a sound receiver that receives reflected sound waves within the coiled wavetube.

In one embodiment, the coiled wavetube is a helical coil.

In one embodiment, the coiled wavetube is a serpentine coil.

In one embodiment, the invention includes a processing system that communicates with the sound receiver for processing the reflected sound.

In one embodiment, the invention includes a display that communicates with and receives data generated by the processing system.

In one embodiment, the apparatus is powered by a battery.

In one embodiment, the apparatus is attached to an ETT.

In one embodiment, the invention includes a wavetube for an acoustic reflectometer in the shape of a coil that conducts sound waves within the wavetube.

In one embodiment, the invention includes a miniaturized acoustic reflectometer that has a wavetube, a sound generator, a sound receiver that communicates with the wavetube, a microprocessor-based processing system that communicates with the sound receiver, and a display that communicates with the processing system in a single unit.

In one embodiment, the wavetube is coiled.

In one embodiment, the display displays an image representative of the cross-sectional area of the surroundings throughout a range of distances beyond the distal end of the wavetube.

In one embodiment, a battery is used to power the reflectometer.

In one embodiment, the invention includes an apparatus for use in an acoustic reflectometer that includes a hermetically-sealed microphone attached within a wavetube. A hermetic seal on the microphone protects the microphone from debris, humidity, and other materials from the area surrounding the acoustic reflectometer that may interfere with the measurements taken by the microphone in the acoustic reflectometer.

These as well as still further features, benefits and advantages of the invention will now become clear from a review of the following detailed description of illustrative embodiments of the invention, taken in conjunction with the accompanying drawings.

### BRIEF DESCRIPTION OF THE DRAWINGS

FIG. 1. is a side elevational view showing an endotracheal tube (“ETT”) improperly inserted into a patient's esophagus.

FIG. 2 is a side elevational view showing an ETT improperly inserted into a patient's bronchus.

FIG. 3 is a side elevational view showing an ETT properly inserted into a patient's trachea.

FIG. 4 is a cut-away and block view of one embodiment of the invention that helps determine the position of the distal end of an ETT in a patient.

FIG. 5 is a graph that is displayed by one embodiment of the invention showing the cross-sectional area beyond the distal end of the ETT in a patient in whom the ETT has been properly inserted into the trachea.

FIG. 6 is a graph that is displayed by one embodiment of the invention showing the cross-sectional area beyond the distal end of the ETT in a patient in whom the ETT has been improperly inserted into the patient's esophagus.

FIG. 7 is a perspective view of one embodiment of the invention having a helical coil, a sound generator, sound receiver, display and energy source.

FIG. 8 is a perspective view of one embodiment of the invention having a serpentine coil, a sound generator, sound receiver, display and energy source.

### DETAILED DESCRIPTION OF THE ILLUSTRATIVE EMBODIMENTS

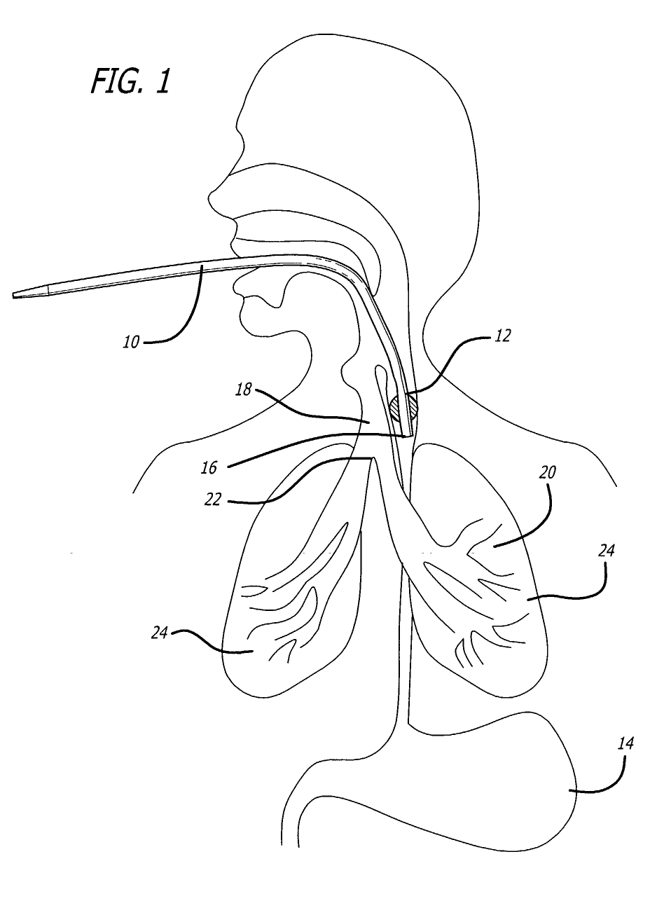

FIGS. 1-3 are side elevational views showing an endotracheal tube (“ETT”) 10 inserted into different anatomical sites in a patient.

FIG. 1 is a side elevational view showing the ETT improperly inserted into the patient's esophagus 12 which, of course, leads to the patient's stomach 14. The distal end 16 of the ETT 10is shown in the esophagus 12 just below the entranceways to the esophagus 12 and trachea 18.

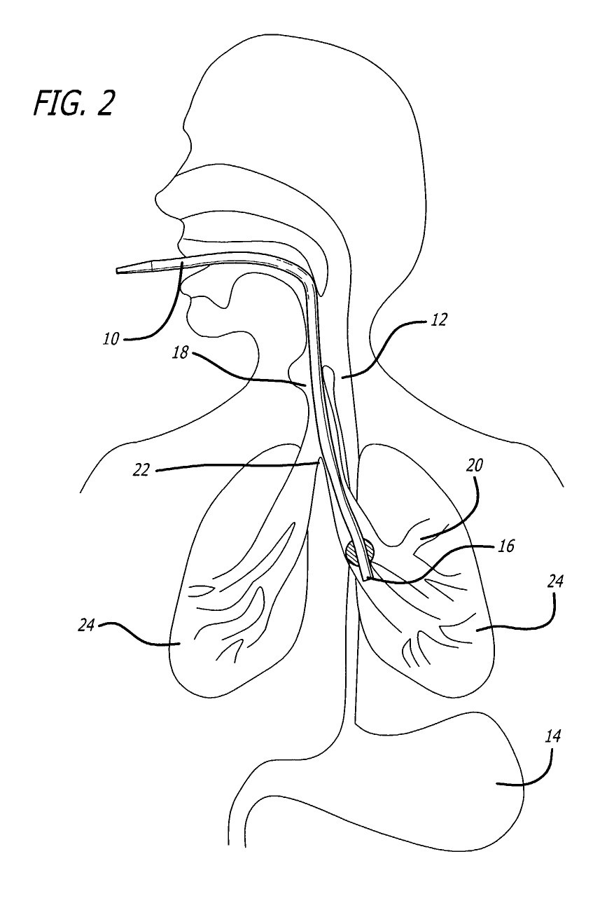

FIG. 2 is a side elevational view showing the ETT improperly inserted into a patient's bronchus 20. An endobronchial intubation such as this occurs when the distal end 16 of an ETT 10 is advanced through a patient's trachea 18, and advanced further beyond the carina 22, into the left (as shown) or right (not shown) mainstem bronchus 20. In such a situation, ventilation of only one of a patient's lungs 24 is possible.

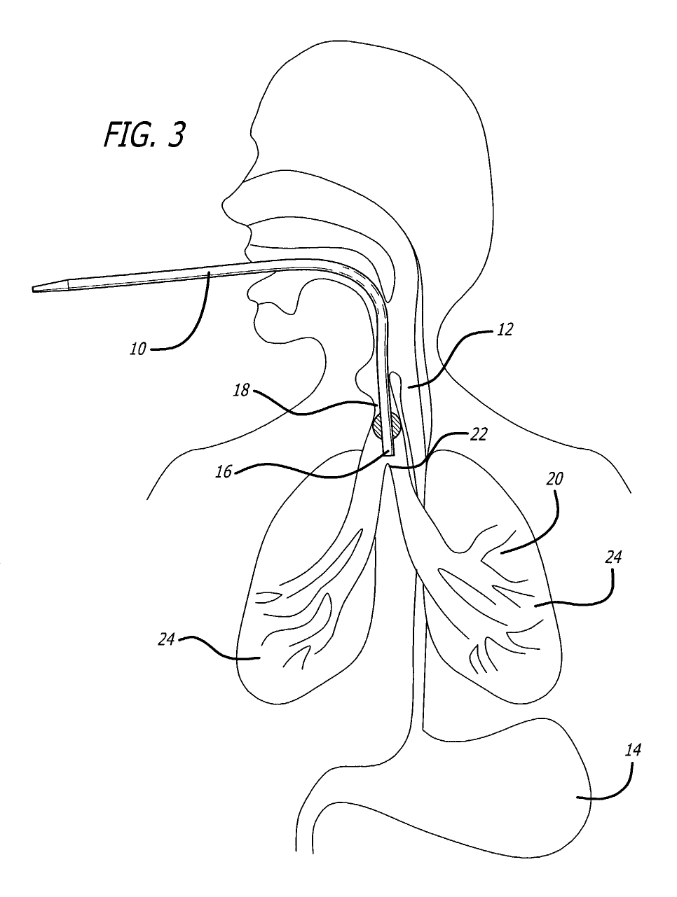

FIG.3 is a side elevational view showing an ETT 10 properly inserted into a patient's trachea 18. The distal end 16 of the ETT 10 is positioned a few centimeters above the carina 22. This allows for the proper ventilation of both of the patient's lungs 24.

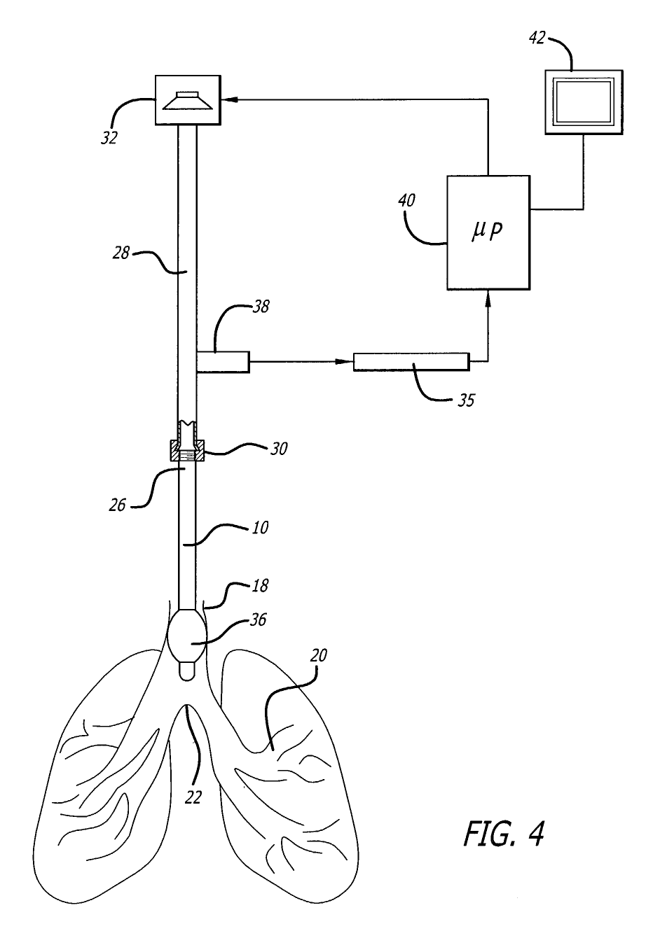

FIG. 4 is a cut-away and block view of one embodiment of the invention that helps determine the position of the distal end 16 of the ETT 10 in a patient using acoustic reflectometry techniques. In this example, the distal end 16 of the ETT 10 is shown properly positioned in the trachea 18.

In one embodiment, the wavetube 28 is connected to the ETT 10 by a connecting adapter 30. In another embodiment (not shown), the wavetube 28 and the ETT 10 are a single, continuous tube. In one embodiment, an ETT cuff 36 is positioned on the distal end 16 of the ETT 10 for a better seal.

The connecting adapter 30, in one embodiment, is substantially sealed and frictionally fitted around a standard ETT adapter that is typically incorporated onto one end of the ETT 10. In one embodiment, the connecting adapter 30 screws tightly onto the standard ETT adapter, thereby acoustically coupling the wavetube 28 to the ETT 10 while creating a seal that prohibits the leakage of sound.

In another embodiment (not shown), the ETT 10 is not equipped with a standard ETT adapter and the connecting adapter 30 is attached directly to the proximal end 26 of the ETT 10. In one embodiment, the connecting adapter 26 snaps onto the proximal end 26 of the ETT 10 creating a seal that prevents the entry or escape of sound from the connected wavetube 28 and ETT 10.

In one embodiment of the invention (not shown), the wavetube 28 and the ETT 10 are attached to a ventilation system. In this embodiment, the patient may be ventilated while the wavetube 28 and ETT 10 are inserted into the patient.

In one embodiment, the adult wavetube 28 has a pre-set internal diameter of approximately 7.0-7.5 mm so as to maximize acoustic transmission at the junction between the wavetube 28 and the approximately same-sized ETT 10. This 7.0-7.5 mm internal diameter wavetube 28 is of a size that is intermediate between those ETT sizes commonly used (8.0-8.5 mm internal diameter for adult mails and 6.5-7.0 mm internal diameter for adult females). The intermediate-sized wavetube 28 is also appropriate for children older than twelve. Additionally, the intermediate-sized wavetube 28 minimizes inventory management demands.

In one embodiment of the invention, the pediatric wavetube 28 has a pre-set internal diameter of approximately 3.0-3.5 mm. The 3.0-3.5 mm internal diameter of ETT 10 is useful with premature infants, who may require an ETT 10 as small as 2.5 mm ETT, and also in larger infants who require a 4.0-4.5 mm internal diameter ETT. Again, inventory management demands are reduced.

In one embodiment of the invention, pulsed sound waves are generated by a sound generator 32 for passage through the wavetube 28, the ETT 10, and the cavity of interest. The sound generator 32, in one embodiment, is electronically driven by a signal generator 40 that generates a series of impulses at the rate of five per second, with each impulse of 2 msec duration. In one embodiment, the impulse spectral frequency range is corresponds to that of a low-pass filter (200-5000 Hz). The sound generator 32 may be located at the proximal end 34 of the wavetube 28 and, in one embodiment, is a loudspeaker. In one embodiment, the sound waves are acoustic pulses. The sound waves typically travel through the wavetube 28 and the ETT 10 to the distal end 16 of the ETT 10.

In the example shown in FIG.4, the distal end 16 of the ETT 10 is in the trachea 18 near the carina 22. When the intubation is done incorrectly, the distal end of the ETT may be in the esophagus 12, as shown in FIG. 1, or improperly in the bronchus, as shown in FIG. 2. In a still further example (not shown), the distal end of the ETT may be obstructed either by a kink, mucus plug, or herniated ETT cuff. On the reflectometer display, obstruction will be manifest on the area-distance profile as an area decrease occurring at a specified axial distance into the cavity. In one embodiment, use of the reflectometer will allow for the early detection and timely interventive treatment of an impending ETT obstruction, thus preventing airway disasters requiring an emergency response. Furthermore, the display will allow for evaluation of the efficacy of the interventive treatment, as reflected by changes in the area profile back toward its normal appearance at the noted axial distance.

The area in the patient beyond the distal end 16 of the ETT 10 will cause the sound waves to be reflected as the cross-section of the area changes. The reflected sound waves return through the distal end 16 of the ETT 10.

In one embodiment, the reflected sound waves are received by a sound receiver 38. The sound receiver 38, in one embodiment, is a single microphone. Another embodiment provides for a sound receiver 38 that is two microphones.

The sound receiver 38 is often exposed to the mucus, bodily secretions, and/or humidified gases of the airway, particularly during exhalations. In one embodiment of the invention, a hermetic seal is placed around the sound receiver 38, such as a hermetic seal around the microphone(s) when used. The hermetic seal protects the sound receiver 38 from ambient particulate or humidified material that can settle on the sound receiver 38. The material may be easily cleaned. The hermetic seal will not usually interfere significantly with the acoustic measurement.

Sound wave signals (reflected acoustic pulses) received by the sound receiver 38 undergo signal processing by the processor 40. In one embodiment, the processor 40 is a micro-processor that processes the digitized electronic equivalent of the amplitudes of the reflected acoustic pulses received by the sound receiver 38. Reflected sound wave signals, in one embodiment, first pass through a low-pass filter 34 before being delivered to the processor 40 from the sound receiver 38. In one embodiment, the processor 40 is programmed to produce data from the sound signal that is representative of the cross-sectional area in the cavity of interest throughout a range of distances beyond the distal end 16 of ETT 10. In one embodiment, the processor 40 uses a Gopillaud-Ware-Aki algorithm to produce the data. Gopillaud-Ware-Aki algorithm is a well known processing technique. More details about the technique can be obtained from Gopillaud PL. “An approach to inverse filtering of near-surface layer effects from seismic records,” Geophysics 1961; 26 (6):754-60; and Ware J. A. & Aki K. “Continuous and discrete inverse-scattering problems in a stratified elastic medium. I: Plane waves at normal incidence,” J. Acoust. Soc. Amer. 1969; 45:911-921, the content of both of which is incorporated by reference herein.

In one embodiment the processor 40 is in communication with a display 42. The display 42 displays a graphic image of the cross-sectional area versus distance-into-the-cavity data that is produced by the processor 40. The graphic images are representative of discontinuities in the medium, through which the acoustic pulses travel, and which are caused by differences in the cross-sectional area beyond the distal end 16 of the ETT 10.

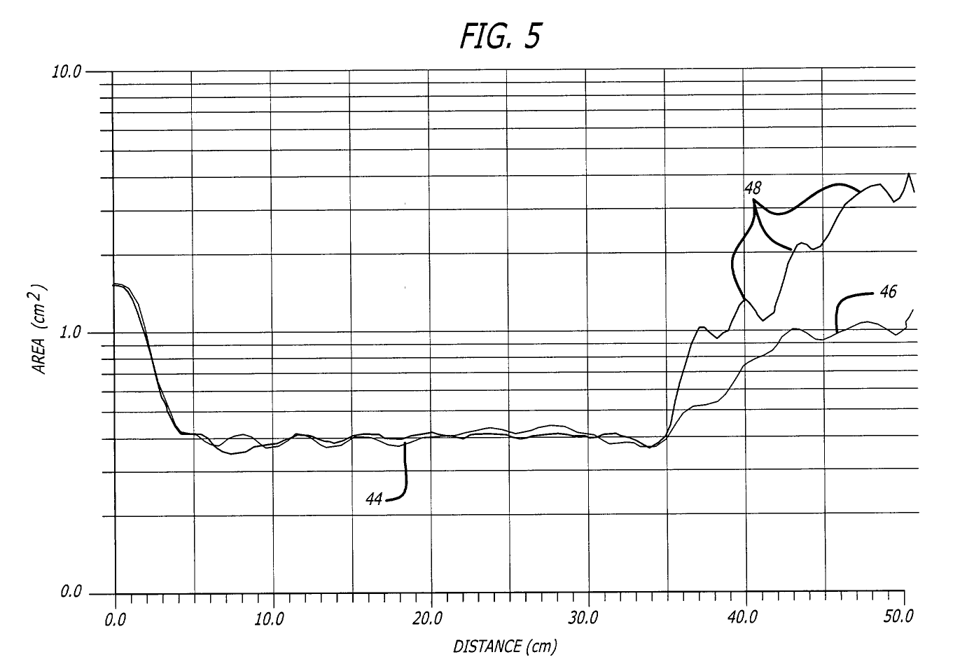

FIG. 5 is a graph that is displayed by one embodiment of the invention showing the cross-sectional area beyond the distal end 16 of the ETT 10 in a patient in whom the ETT has been properly inserted into the patient's trachea. A characteristic feature of the graph is a constant cross-sectional area segment 44 corresponding to the length of the ETT 10. After the segment 44 is a rapid rise 48, representing a rapid increase in the airway area beyond the distal end 16 of the ETT 10. This is followed by a slower rise, representing a further increase in the cross-sectional area at further distances into the lung.

With proper training, the physician will interpret the graph shown in FIG. 5 to represent a correct tracheal intubation. The segment 44 represents the constant cross-sectional area segment corresponding to the length of the ETT. The peaks and troughs in 46 are caused by the single bifurcation at the carina and by the multiple successive bifurcations deeper within the lungs 24.

An erroneous endobronchial intubation results in a graph similar to that of the lower trace 46 in FIG. 5; this results from a malpositioning of the ETT. However, the area rise 46, which represents the cross-sectional airway area beyond the carina 22, is only about one-half of the total area shown in the upper trace 48 of FIG. 5. In the case of an endobronchial intubation, proper repositioning requires that the tube be withdrawn into the trachea until the full area trace of 48 is restored. The physician thus analyzes and interprets the area-versus-distance graph to determine the position of the ETT 10 in the patient.

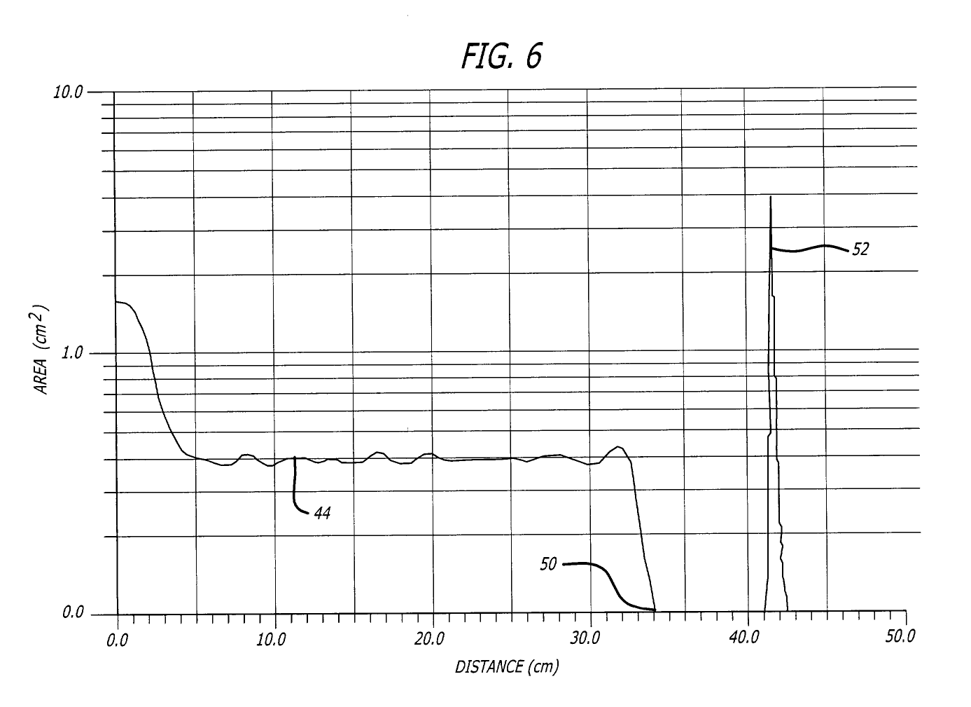

FIG. 6 is a graph that is displayed by one embodiment of the invention showing in a patient the cross-sectional airway area through a distance beyond the distal end 16 of the ETT 10 when the ETT 10 has been improperly inserted into the patient's esophagus 12. When the distal end 16 of the ETT 10 is in the esophagus 12, the compliant walls of the esophagus 12 close around the distal end 16 of the ETT 10. The closed esophageal lumen prevents further transmission of the acoustic impulse down the cavity, causing the graph to drop to about zero at segment 50. Thus, when the distal end 16 of the ETT 10 is in the esophagus 16, the characteristic graph consists of a segment of constant cross-sectional area 44 corresponding to the length of the ETT 10, followed by a sharp decrease 50 in the cross-sectional area to about zero. This decrease 50 occurs because the esophageal lumen is ordinarily closed immediately beyond the tip of the ETT 10. The graph of an esophageal intubation may also include a stray spiked artifact 52 that is present beyond the zero-area axial length segment 50.

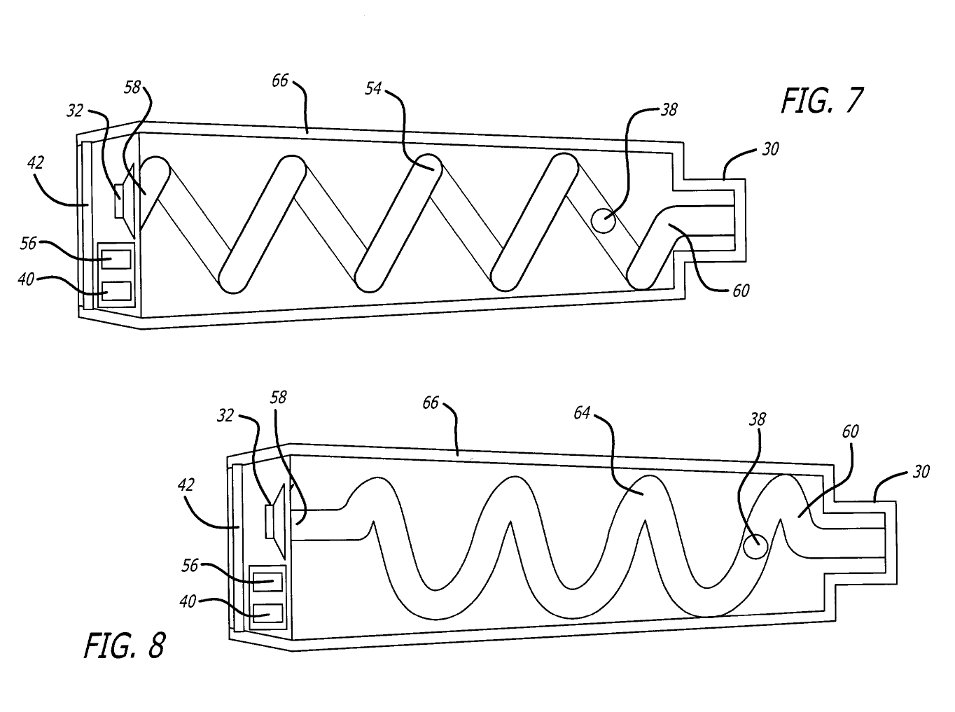

FIG. 7 is a perspective view of one embodiment of the invention having a helical coil 54, a sound generator 32, sound receiver 38, display 42 and energy source 56. The helical coil 54 functions as the wavetube 28 of the invention. It permits an overall reduction in the length of the wavetube in the axial direction of the ETT 28, thus allowing for a much smaller and compact acoustic reflectometer. All of these components may be encased in a casing 66. The miniaturization of the reflectometer also reduces junctional area mismatching.

Near the proximal end 58 of the helical coil 54 is the sound generator 32 for generating sound waves. The sound generator 32 may be a loudspeaker.

The sound waves generated by the sound generator 32 travel through the helical coil 54 to the distal end 60 of the helical coil 54. The distal end 60 is then connected to the ETT, such as the ETT 10 shown in FIGS. 1-4. Sound waves that are reflected from the area beyond the distal end 60 of the helical coil 54 return through the distal end 60 of the helical coil 54. The sound waves are received by the sound receiver 38, preferably at the distal end 60 of the helical coil 54. The sound receiver 38 is preferably a single microphone. The sound receiver 38 also preferably has a hermetic seal (not shown).

Sound wave signals generated by the sound receiver 38 are processed by a processor 40. The processed sound waves are delivered to and displayed on the display 42 which, in one embodiment, is located at the proximal end 58 of the helical coil. The display 40, in one embodiment, is a small screen. In another embodiment, this display 40 is easily detachable from the helical coil 54. The display 40 in one embodiment displays the graph of distance versus the cross-sectional area, such as the graph shown in FIG. 5 or FIG. 6.

A free-standing energy source 56, such as a lithium iodide battery, may also be used to power the invention. If the battery becomes exhausted, it may be replaced. In an alternate embodiment, the acoustic reflectometer may be externally powered. An AC-DC converter cable connected to an AC outlet and a portable defibrillator may also advantageously be used.

FIG. 8 is a perspective view of another embodiment of the invention. It is identical to FIG. 7, except that the wavetube 64 is a serpentine, S-shaped coil 62.

As with the helical coil wavetube, the effect of the serpentine, S-shaped coil is to reduce the effective length of the wavetube in the axial direction of the wavetube. It too permits an overall reduction in the length of the wavetube in the axial direction of the ETT 28, thus allowing for a much smaller and compact acoustic reflectometer.

Both the helical coil 54 and the serpentine coil 64 wavetubes may be of different lengths, depending on the purpose for which the acoustic reflectometer is being used. In one embodiment, the helical and serpentine coils 54 and 64 are platinum-cured. In one embodiment, the helical coil 54 and the serpentine coil 64 are made of a silicon material which can be cut to any desired length. In a still further embodiment, the helical coil 54 and serpentine coil 64 are made of material that is affordable to dispose after a single use. In one embodiment, the wavetube is disposed along with the sound receiver 38, while in another embodiment, the sound receiver 38 is detachable and only the wavetube 28 is disposed after the single use.

While particular embodiments of the invention have now been described, those of ordinary skill will readily devise variations of the present invention without departing from the inventive concept. For example, when determining the placement of an ETT 10, the processor 40 and the display 42 may be coupled to additional diagnostic devices thereby allowing the clinician to evaluate several variables of the patient's condition at once. The coiled wavetubes 54 and 64, the hermetically-sealed sound receiver 38, and the miniature embodiments of the invention illustrated in FIGS. 7 and 8 may be used in a variety of acoustic reflectometer devices and applications, not just the clinical settings discussed herein.

I claim:

1. A method for determining the position inside of a patient of an endotracheal intubation using an endotracheal tube having a proximal and a distal end comprising:
a) inserting the distal end of the endotracheal tube through the mouth or nose of a patient;
b) sending sound waves through a wavetube in acoustic communication with the endotracheal tube and with the body cavity of the patient;
c) receiving reflections of the sound waves within the wavetube;
d) transforming the reflections into data representative of the cross-sectional area of the body cavity in the patient throughout a range of distances beyond the distal end of the endotracheal tube;
e) displaying an image of the cross-sectional area of the body cavity in the patient throughout the range of distances; and
f) examining the display to determine the position of the distal end of the endotracheal tube within the patient.

2. The method of claim 1 wherein said transforming the reflections includes applying a Gopillaud-Ware-Aki algorithmic calculation.

3. The method of claim 1 wherein the distal end of the endotracheal tube at the time of receiving is near the carina and wherein the image includes a length of constant cross-sectional area of the body cavity followed by a length of rapid increase in the area.

4. The method of claim I wherein the distal end of the endotracheal tube at the time of the receiving is in the esophagus and wherein the image includes a length of constant cross-sectional area of the body cavity followed by a length with a decrease in the area to approximately zero.

5. The method of claim 4 further comprising at least partially removing the endotracheal tube after said examining and re-inserting the endotracheal tube in the trachea.

6. The method of claim 4 further comprising delivering oxygen through the endotracheal tube after determining proper positioning of the endotracheal tube in the patient.

7. The method of claim 1 wherein the distal end of the endotracheal tube at the time of the receiving is in the bronchus and wherein the image includes a length of constant cross-sectional area of the body cavity, followed by a length of a moderate decrease of approximately 50 % or more of the total area on the area-distance profile relative to an area image when the endotracheal tube is in the trachea.

8. The method of claim 7 further comprising at least partially removing the endotracheal tube from the bronchus, and re-inserting the endotracheal tube in the trachea to the area above and near the carina.

9. The method of claim 1 further comprising connecting the endotracheal tube and the wavetube with a connecting adapter.

10 The method of claim 9 wherein the endotracheal tube has an endotracheal tube adapter on one end and the connecting adapter is frictionally fitted onto the endotracheal tube adapter.

11. The method of claim 10 wherein said sound waves include acoustic pulses less than two milliseconds in length.

12. The method of claim 1 wherein said sound waves include a series of pulses.

13. The method of claim 1 wherein said sound waves include a series of reflected pulses and wherein said transforming includes averaging the reflected series of pulses.

14. The method of claim 1 wherein said sending sound waves includes delivering the sound waves via a loudspeaker.

15. The method of claim 1 wherein a single microphone is used for said receiving reflections of the sound waves.

16. The method of claim 1 wherein two microphones are used for said receiving reflections of the sound waves.

17. The method of claim 1 further comprising disposing of the wavetube after using the wavetube with just one patient.

18. The method of claim 1 further comprising disposing of the endotracheal tube after using the endotracheal tube with just one patient.

19. The method of claim 1 wherein a sound receiver is used during said receiving and further comprising disposing of the sound receiver after using the sound receiver with just one patient.

20. The method of claim 1 further comprising ventilating the patient through the wavetube and the endotracheal tube.

21. The method of claim 1 wherein the endotracheal tube and the wavetube are a single tube.

22. The method of claim 21 further comprising ventilating the patient through the single tube.

23. An instrument for performing an endotracheal intubation in a patient comprising:
a) an endotracheal tube having a proximal and a distal end, wherein the distal end is inserted through the mouth or nose of a patient;
b) a wavetube for acoustic communication with said endotracheal tube;
c) a sound generator for generating sound for delivery through said wave tube and said endotracheal tube;
d) a sound receiver for receiving reflections of the sound within the wavetube and the body cavity;
e) a processor in communication with said sound receiver for transforming the reflections into data representative of the cross-sectional area of the body cavity in the patient throughout a range of distances beyond the distal end of the endotracheal tube; and
f) a display in communication with said processor for displaying an image of the cross-sectional area of the body cavity in the patient throughout the range of distances.

24. The instrument of claim 23 wherein said wavetube is made of silicon.

25. The instrument of claim 23 wherein said sound generator includes a loudspeaker.

26. The instrument of claim 23 wherein said sound receiver includes only one microphone.

27. The instrument of claim 26 wherein said microphone is hermetically-sealed.

28. The instrument of claim 23 wherein said sound receiver includes two microphones.

30. The instrument of claim 28 wherein said microphones are hermetically-sealed.

32. The instrument of claim 23 further comprising a connecting adapter for connecting said wavetube to said endotracheal tube.

33. The instrument of claim 32 wherein said endotracheal tube has an endotracheal tube adapter on one end and said connecting adapter is frictionally fitted onto said endotracheal tube adapter.

34. The instrument of claim 23 wherein said display is attached to said wavetube in a manner that is easy for the user to detach.

35. The instrument of claim 23 further comprising a battery for providing power.

36. The instrument of claim 23 wherein said wavetube has an internal diameter of approximately 7.0-7.5 mm and is adapted for imaging a body cavity in an adult human patient.

37. The instrument of claim 23 wherein said wavetube has an internal diameter of approximately 3.0-3.5 mm and is adapted for imaging a body cavity in a pediatric human patient.

38. The instrument of claim 23 wherein said wavetube has an inner cross-sectional area of no more than 0.5 square centimeters and is adapted for imaging an area in an adult human patient.

39. The instrument of claim 23 wherein said wavetube has an inner cross-sectional area of no less than 0.07 square centimeters and is adapted for imaging an area in a pediatric human patient.

40. The instrument of claim 23 wherein said wavetube is coiled.

41. The instrument of claim 40 wherein said wavetube is coiled in a helical configuration.

42. The instrument of claim 40 wherein said wavetube is coiled in a serpentine configuration.

43. The instrument of claim 23 wherein said endotracheal tube and said wavetube are a single tube.

44. Apparatus for use with an acoustic reflectometer comprising:
a) a coiled wavetube for communicating acoustic signals;
b) a sound generator in communication with said coiled wavetube for generating sound waves in said coiled wavetube; and
c) a sound receiver in communication with said coiled wavetube for receiving reflections of the sound waves within said coiled wavetube.

45. The apparatus of claim 44 wherein said sound generator includes a loudspeaker.

46. The apparatus of claim 44 wherein said sound receiver includes only one microphone.

47. The apparatus of claim 44 wherein said sound receiver includes two microphones.

48. The apparatus of claim 44 further comprising a processing system in communication with said sound receiver for processing the reflected sound.

49. The apparatus of claim 44 further comprising a display in communication with said processing system.

50. The apparatus of claim 44 wherein said coiled wavetube has a proximal end and wherein said display is positioned adjacent to said proximal said end.

51. The apparatus of claim 44 further comprising a battery for providing power.

52. The apparatus of claim 51 wherein said coiled wavetube, sound generator, sound receiver, processing system, display and battery are encased together.

53. The apparatus of claim 44 wherein said coiled wavetube is substantially helical in shape.

54. The apparatus of claim 44 wherein said coiled wavetube is substantially serpentine in shape.

55. The apparatus of claim 44 wherein said coiled wavetube has a distal end configured to attach to an endotracheal tube.

56. The apparatus of claim 44 wherein said coiled wavetube has a distal end that is integral to an endotracheal tube.

57. A wavetube for an acoustic reflectometer comprising a tube for conducting sound waves within it that is shaped in a coil.

58. An integrated and miniaturized acoustic reflectometer comprising:
a) a wavetube having a proximal and a distal end;
b) a sound generator in acoustic communication with said wavetube;
c) a sound receiver in acoustic communication with said wavetube;
d) a microprocessor-based processing system in communication with said sound receiver; and
e) a display in communication with said processing system,
f) wherein said wavetube, sound generator, sound receiver, processing system and display form an integral unit.

59. The relectometer of claim 58 wherein said display is adjacent to the proximal end of said wavetube.

60. The relectometer of claim 58 wherein said sound receiver includes a microphone attached to said wavetube.

61. The relectometer of claim 58 wherein said wavetube is coiled.

62. The relectometer of claim 61 wherein said wavetube is coiled in a serpentine configuration.

63. The reflectometer of claim 61 wherein said wavetube is coiled in a helical configuration.

64. The reflectometer of claim 58 wherein said processing system and said display are configured to cause the display of an image representative of the cross-sectional area of the surroundings throughout a range of distances beyond the distal end of said wavetube.

65. The reflectometer of claim 58 further comprising a battery within said integral unit for providing power.

66. Apparatus for use in an acoustic reflectometer comprising:
a) a wavetube;
b) a microphone attached to said wavetube; and
c) a hermetic seal on said microphone to protect said microphone.
